(19) United States
(12) Patent Application Publication
(10) Pub. No.: US 2006/0194194 A1
(43) Pub. Date: Aug. 31, 2006

(54) Diagnostic measurement of disease

(76) Inventors: Michael Mayer, Ann Arbor, MI (US); Sohiel Memarsadaghi, Ann Arbor, MI (US); Daniel J. Estes, Nashville, TN (US)

Correspondence Address: HARNESS, DICKEY & PIERCE, P.L.C., P.O. BOX 828, BLOOMFIELD HILLS, MI, 48303, US

(21) Appl. No.: 11/352,081

(22) Filed: Feb. 10, 2006

Related U.S. Application Data
(60) Provisional application No. 60/652,030, filed on Feb. 11, 2005.

(51) Int. Cl. C12Q 1/00; C07K 14/705

(52) U.S. Cl. 435/4

(57) ABSTRACT

Diagnostic systems and methods that can be used to perform direct tests on immune system or other proliferative cells, which are capable of identifying abnormal cell function associate with disease without use of labeled reagents; that also permit, but do not require provision of an autoantigen; that are capable of routine use; and that can be used to make early detection of autoimmune diseases or disorders, of epitope spreading pursuant to an autoimmune disease or disorder, or of non-autoimmune proliferative diseases or disorders.

### CROSS-REFERENCE TO RELATED APPLICATIONS

This application claims the benefit of U.S. Provisional Application No. 60/652,030, filed on Feb. 11, 2005. The disclosure of the above application is incorporated herein by reference.

### FIELD

The present teachings relates to methods and systems for diagnosing and monitoring autoimmune diseases and disorders and non-autoimmune proliferative diseases and disorders.

### BACKGROUND

The statements in this section merely provide background information related to the present disclosure and may not constitute prior art.

Autoimmune diseases and disorders, and non-autoimmune proliferative diseases and disorders, are typically diagnosed after they have become established in a subject. This often involves, e.g., an imaging technique to visualize either a suspected site of tissue degradation or inflammation, or a suspected tumor site in the body. Such tests are normally performed for patients already presenting with health complaints.

Biopsies are typically performed to obtain tissue from such patients for diagnostic assay, in order to generate a basis for diagnosis. Autoimmune diagnostic assays traditionally involve assays of, e.g., serum proteins and factors, such as antibodies and complement inhibitors. Typical diagnostic assays involve detection of a subject's autoantibodies or of the subject's antibody protein concentration ratio(s), the latter typically determining a subject's IgG or IgA subclass deficiencies or IgG light chain subclass deficiencies. In the case of autoantibodies, binding assays are performed either: (1) using detectably labeled reagents, such as detectably labeled antigens or detectably labeled anti-idiotypic antibodies in immunofluorescence or enzyme immunoassays for detection of a labeled binding reaction product; or (2) using immunodiffusion techniques or spectrophotometric antibody-antigen binding tests.

However, such tests rely on measurement of secondary factors, such as immune system products, rather than on direct measurements of immune system components. In part because of this, the assays are attendant with a potential risk of missed diagnosis or misdiagnosis because of, e.g., antigen cross-reactivity and patient-specific biochemical variations, and because in some cases, autoantibodies cannot be detected from a biopsied sample, as a result of cross-reaction in vivo with the patient's own anti-idiotypic antibodies in a phenomenon called masking. Such assays are also limited by the choice and availability of autoantigens or anti-idiotypic antibodies to be used. In addition, the assays typically lack the capability to perform early detection of disease or early detection of epitope spreading pursuant to the disease.

Moreover, not every autoimmune disease has yet had its target antigen identified, and it is likely that there are a number of diseases not yet recognized as being autoimmune in nature. For example, it was not until 1988 that type 1 diabetes was identified as an autoimmune disorder, and only since then have vitiligo and psoriasis been so identified.

As a result, it would be advantageous to provide a diagnostic system and method: that can be used to perform direct tests on immune system or other proliferative cells; that are capable of identifying abnormal cell function associated with disease without use of labeled reagents; that also permit, but do not require, provision of an autoantigen; that are capable of routine use; and that can be used to make early detection of autoimmune diseases or disorders, of epitope spreading pursuant to an autoimmune disease or disorder, or of non-autoimmune proliferative diseases or disorders.

### SUMMARY

According to the principles of the present teachings, various embodiments diagnostic systems and methods that can be used to perform direct tests on immune system or other proliferative cells, which are capable of identifying abnormal cell function associate with disease without use of labeled reagents; that also permit, but do not require provision of an autoantigen; that are capable of routine use; and that can be used to make early detection of autoimmune diseases or disorders, of epitope spreading pursuant to an autoimmune disease or disorder, or of non-autoimmune proliferative diseases or disorders.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 shows a cartoon illustrating the involvement of voltage-gated Kv1.3 channels and voltage-independent Ca2′ release-activated Ca²⁺ (CRAC) channels in the activation of a CD4⁺ T cells by an antigen-presenting cell (APC).

FIG. 2 shows the principle of high throughput ion channel recording. A) Schematic side-view of an individual microwell with a cell sealed automatically onto a small pore by suction. B) Schematic top-view of a section of 20 microwells. C) Photograph of a 384-well “patch-plate” used in the instrument from Essen Instruments. D) Photograph of the Ion Works High-Throughput ion-channel analysis instrument.

FIG. 3 shows the measure of specificity improvement when testing for ion-gated channels using T-cells enriched for CD8+.

FIG. 4 shows the comparison of Kv1.3 activity in MS patients and control subjects from CD8⁺ enriched T cell preparations. Applying the same experimental protocol and analysis to all samples. Kv1.3 activity is determined by the instrument. Only cells with a seal resistance of at least 75 MOhms are included in the analysis.

FIG. 5 shows the comparison of the total Kv1.3 current in MS patients and control subjects from three different T cell preparations. The total Kv1.3 currents in MS patients are normalized to the currents found in the respective preparations of control subjects (the average total current for each T cell preparation in control and subjects was set to 100%).

FIG. 6 shows the comparison of ion channel activity in lymphocytes from MS patients with control patients. The bar-graphs indicate that blood samples from M S patients contained up to 16% cells with “MS-specific Kv1.3 ion channel currents” (activity>150 nA·nsec).

FIG. 7 shows representative traces of Kv1.3 current in patient and control subjects before and after addition of ShK blocker.

### DETAILED DESCRIPTION

The following description is merely exemplary in nature and is not intended to limit the present disclosure, application, or uses.

Various embodiments of the present invention provide clinically useful diagnostic tests and systems that involve measurement of ligand and voltage-gated ion channel activity. This novel approach to clinical measurement and diagnosis of disease permits the clinician to directly measure immune cell or other proliferative cell activity and, thus, provides a basis for making more consistently accurate assessments of the development, presence, or state of non-autoimmune proliferative, or of autoimmune, diseases or disorders. Early detection of disease or disease progression are thus made possible for numerous diseases and disorders.

In various embodiments, methods hereof involve measuring ion flux across voltage-gated ion channels. Such ion channels are at least partly responsible for the electrical potential of any cell, the potential being generated and maintained by controlling the movement of ions across the plasma membrane. This movement of ions requires ion channels, which form ion-selective pores within the membrane. There are two recognized major classes of ion channels: ion transporters and gated ion channels. Ion transporters utilize energy obtained from ATP hydrolysis to actively transport an ion against the ion's concentration gradient. Gated ion channels allow passive flow of an ion down the ion's electrochemical gradient under restricted conditions. Together, these types of ion channels generate, maintain, and utilize an electrochemical gradient that is used in 1) electrical impulse conduction down the axon of a nerve cell, 2) transport of molecules into cells against concentration gradients, 3) initiation of muscle contraction, and 4) endocrine cell secretion.

In various embodiments, methods hereof involve measurement of voltage-gated potassium ion channels. Potassium channel subunits of the Shaker-like superfamily all have the characteristic six transmembrane/1 pore domain structure. Four subunits combine as homo- or heterotetramers to form functional K channels. These pore-forming subunits also associate with various cytoplasmic beta subunits that alter channel inactivation kinetics. The Shaker-like channel family includes the voltage-gated K⁺ channels as well as the delayed rectifier type channels such as the human ether-a-go-go related gene (HERG) associated with long QT, a cardiac dysrythmia syndrome (Curran, M. E. (1998) Curr. Opin. Biotechnol. 9:565-572; Kaczorowski, G. J. and M. L. Garcia (1999) Curr. Opin. Chem. Biol. 3:448-458).

In various embodiments, methods hereof involve measurement of voltage-gated ion channels from lymphocytes, such as T-cells. T cells are divided into two major groups, CD4+ T helper (Th) cells, and CD8+ cytotoxic T lymphocytes (CTL). Immune responses are primarily regulated by CD4+ Th cells, which fall into two subclasses based on the kinds of cytokines they secrete. Th1 cells secrete mainly interferon-gamma and interleukin (IL)-2, regulates the responses of CTLs, B cells, and macrophages, and orchestrates the removal of intracellular pathogens. In contrast, Th2 cells secrete primarily IL-4 and IL-10 and promote the development of certain antibody responses such as IgG1, IgA, and IgE. An excess of IgE triggers allergic responses. By enhancing the antibody response, Th2 cells regulate removal of extracellular pathogens including various bacteria and parasites.

Abnormal regulation of T-cell activation is involved in a number of diseases, disorders, and conditions of the immune system. Organ-specific autoimmune disorders, such as insulin-dependent diabetes mellitus, multiple sclerosis, rheumatoid arthritis, systemic lupus erythematosus, chronic graft versus host disease, and Crohn's disease result from an abnormal T-cell response to self tissues. T-cells also mediate allergic and other atopic immune responses. Such diseases and conditions may be detected for diagnosis or monitoring, or for disease-specific therapy development or patient-specific therapy titration, according to some embodiments of the present invention.

Various embodiments of the present invention can employ a patch plate, rather than traditional patch clamp, technique, the traditional patch clamp technique not being robust enough to permit routine clinical use or a sufficiently efficient process to permit diagnosis based on measurement of a statistically meaningful patient cell population.

The traditional patch clamp technique allows measurement of ion flow through ion channel proteins and the analysis of the effect of drugs on ion channels function. In brief, in the standard patch clamp technique, a thin glass pipette is heated and pulled until it breaks, forming a very thin (<1 μm in diameter) opening at the tip. The pipette is filled with salt solution approximating the intracellular ionic composition of the cell. A metal electrode is inserted into the large end of the pipette, and connected to associated electronics. The tip of the patch pipette is pressed against the surface of the cell membrane. The pipette tip seals tightly to the cell and isolates a few ion channel proteins in a tiny patch of membrane. The activity of these channels can be measured electrically (single channel recording) or, alternatively, the patch can be ruptured allowing measurements of the combined channel activity of the entire cell membrane (whole cell recording).

During both single channel recording and whole-cell recording, the activity of individual channel subtypes can be further resolved by imposing a voltage clamp across the membrane. Through the use of a feedback loop, the voltage clamp can impose a user-specified potential difference across the membrane, allowing measurement of the voltage, ion, and time dependencies of various ion channel currents. These methods allow resolution of discrete ion channel subtypes.

A major limitation of the patch clamp technique as a general method in pharmacological screening is its low throughput. Typically, a single, highly trained operator can test fewer than ten compounds per day using the patch clamp technique. Furthermore the technique is not easily amenable to automation, and produces complex results that require extensive analysis by skilled electrophysiologists.

As a result, before the present invention, no clinically useful system or method for disease diagnosis has been provided that is based on voltage-gated ion channel measurement or profiling, in spite of the potential for gated ion channel profiling to improve diagnosis, prognosis, and treatment of disease. For example, both the levels and types of gated ion channels expressed in autoreactive memory T lymphocytes from subjects with autoimmune disease may be compared with the levels and sequences expressed in T-cells from normal subjects.

Pursuant to various embodiments according to the present teachings, it is now possible to clinically screen patient tissues, e.g., white blood cells, using a sensitive electrophysiological and a flow cytometry based fluorescence method that detects pathologic ion channel activity. This can now permit the clinician more conclusively and earlier to diagnose diseases such as autoimmune, graft versus host, cardiac, neuronal and cancer, and to monitor the success of a given therapy by correlating it to ion channel activity.

After separation as described herein to obtain CD4+ and CD8+ from human tissue, the lymphocytes can be incubated with a fluorescently-labeled molecule that specifically binds to Kv1.3 ion channels (for example but not limited to fluorescently-labeled ShK), such as for about 30-45 min. After wash steps (e.g. 3×) in an appropriate buffer (e.g. 98% dPBS/2% NCS), flow cytometry can be run to quantify the amount of channel blocker on the surface of the cell. The channel blocker can be fluorescently labeled on a variety of subsets of lymphocytes. Specific subsets of lymphocytes can also be selected using fluorescently labeled antibodies to cell-surface markers (e.g. effector memory T cells by selecting for cells negative for CCR7 and CD45RA). Certain subsets of cells in diseased patients exhibit higher amounts of fluorescence due to the labeled channel blocker. The presence of increased fluorescence due to the channel blocker indicates that that particular cell overexpresses the specified ion channel. In some embodiments, by comparing the fluorescence levels of channel blocker in controls versus diseased patients, flow cytometry can provide a basis for an assay for auto-immune diseases.

In some embodiments hereof, high throughput screening assays are provided that can be used to diagnose specific disease states that are associated with aberration in voltage-gated ion channel conductance. High throughput is the capability to measure the electrophysiological activity of ion channel activity in at least 10 cells, at least 20 cells, at least 40 cells, at least 50 cells, at least 100 cells, at least 200 cells, at least 500 cells, or, in some embodiments, at least 1000 cells sequentially, or concurrently (e.g. in parallel); in the context of measuring the activity of, e.g., a single voltage gated ion channel containing structure, high throughput means that the total time involved both in contacting the cells, cell fragments or cell membranes to be analyzed, with the electrodes in such a manner that the electrophysiological measurement can be made, and in obtaining such a measurement, is about 3 minutes or less, or about 150 seconds or less, or about 2 minutes or less, or about 90 seconds or less, or about 60 seconds or less; in some embodiments, the total time can be about 45 seconds or less. In some embodiments such a total time can be involved in measuring each ICC structure in a series of ICC structures being measured sequentially. In some embodiments, screening of one or more patient's and control's cellular ion channel activity can be accomplished in high throughput, wherein the screen is capable of multiplexing up to 384 recording elements to a data acquisition/processor system utilizing multiple voltage-clamp amplifiers. This has the unexpected advantage of providing the highest known resolution and effectively simultaneous measurement from all wells. This provides the added advantage that the IonWorks HT (High Throughput) electrophysiology apparatus with the potential to achieve throughput rates on par with the most productive fluorescence based ligand-receptor binding assays (≧150,000 compounds per week).

With regard to high throughput screening of ion channels on whole cells, cell fragments, cellular membranes and combinations thereof, at least 5 cells but less than 20 cells, at least 10 cells but less than 40 cells, at least 15 cells but less than 100 cells, at least 20 cells but less than 1000 cells can be analyzed using automated patch plate electrophysiological analysis in parallel. High throughput analysis is required, even for one patient sample because the target cells to be screened are commonly found in very low numbers. Therefore, to make definitive and reasoned diagnostic evaluation of the relationship between disease and disorder and healthy on the basis of ion channel activity, statistical analysis would require large sample numbers.

Disorder and disease is generally defined as an impairment of the normal state of the living animal body or one of its parts that interrupts or modifies the performance of the vital functions, is can be manifested by distinguishing signs and symptoms, and is a response to environmental factors (as malnutrition, industrial hazards, or climate), to specific infective agents (as worms, bacteria, or viruses), to inherent defects of the organism (as genetic anomalies), or to combinations of these factors. In other words, the sum of the entire ion channel current per unit of blood could serve as the metric for distinguishing between those with a disorder or disease and those who do not have the same disorder or disease. Disorders and diseases are defined herein as those disorders and conditions that can be diagnosed and monitored by finding a statistically significant increase in total ion channel activity as compared to controls that do not have the disorder or disease, when tested using the same clinical procedures.

The present invention now proposes and allows for the first time, screening methods based on defined cell populations taken from patients and asymptomatic individuals that measure the number and activity of gated ion channels that are intimately associated with disease which can be performed on large samples of individual cells in parallel providing increased sensitivity, specificity, reliability and a higher throughput. The present invention provides for methods describing high throughput ion channel screening in blood cells for diagnosis and therapeutic monitoring of subjects with autoimmune disease, cardiac, skeletal, cancer, chronic graft versus host or infectious diseases and to assess the efficacy of therapeutic drugs and vaccines on these conditions.

In some embodiments, the methods can be used to diagnose the presence of disease by measuring the differential expression pattern and activity of voltage gated ion channels in patients suspected of having a proliferative or immunological disease but not presenting clinical symptoms of such diseases by comparing the differential expression pattern and activity of voltage gated ion channels between the patient and healthy controls.

In some embodiments of the present invention cells expressing Kv1.3 potassium ion channels are screened for differential expression and activity using an electrophysiological recording device. In general, inhibition of K⁺ channel function leads to a decrease in proliferation both in models in which proliferation is a physiological response (the case of lymphocytes) and in those in which it is a manifestation of a pathological condition (as in cancer cells). Of the relevant potassium channels used to diagnose and monitor the presence of autoimmune or proliferative disease, these include the Shaker family of voltage-gated potassium ion channels. Kv1.3 is the dominant channel in resting T cells. Kv10.1 and Kv1.1 have been implicated in proliferation processes, mainly in tumor proliferation. Although examples are provided herein for the use in methods of diagnosing and monitoring diseases related to autoimmune and proliferative disorders involving Kv1.3, other known calcium and potassium ion channels which are upregulated and over expressed can be used as selective markers of autoimmune and proliferative disorders. Several known potassium channels, such as those disclosed in Table 2 in U.S. Pat. No. 6,686,193 are useful as selective markers for screening ion channel activity and are incorporated by reference herein.

Though not bound by theory, it is believed that other voltage regulated potassium channels, such as other Kv1, Kv2, Kv3, Kv4, Kv5, Kv6, Kv7, Kv8, and Kv9 channels, or at least any of the Kv1 channels, also exhibit a like response as do Kv1.3 channels and can likewise be used, by identification of an increase in their specific ion channel activity or by identification of an increase in both their specific ion channel activity and prevalence per cell, to detect cells exhibiting a differential ion channel expression pattern and thereby detect cell states associated with the development of, presence of, or status of, a disease or disorder. The detected result can be compared against a standard or control result, obtained for healthy cells, to help a medical or veterinary practitioner diagnose the development of, diagnose the presence of, or to monitor the status of an associated disorder or disease. For example, Kv1 subtypes have been found to be highly conserved in the primary, secondary, and tertiary structures of their polypeptides and in their quaternary structures. See, e.g., H-L Liu et al., Homology Models of the Tetramerization Domain of Six Eukaryotic Voltage-gated Potassium Channels Kv1.1-Kv1.6, J. Biomolec. Str. & Dyn. 22(4):388-98 (February 2005).

In some embodiments the present screening assays measure the activity of voltage gated ion channel activity when disease specific effector cells for example, neoplastic B-cells (lymphoma), T-lymphocytes (including late memory effector cells) from patients suspected or confirmed with diabetes mellitus, Multiple Sclerosis (MS), rheumatoid arthritis, psoriasis, Crohns Disease and chronic graft-vs-host disease are stimulated with antigens and that are known to upregulate cytokine production and/or increase uncontrolled division and escape immune surveillance.

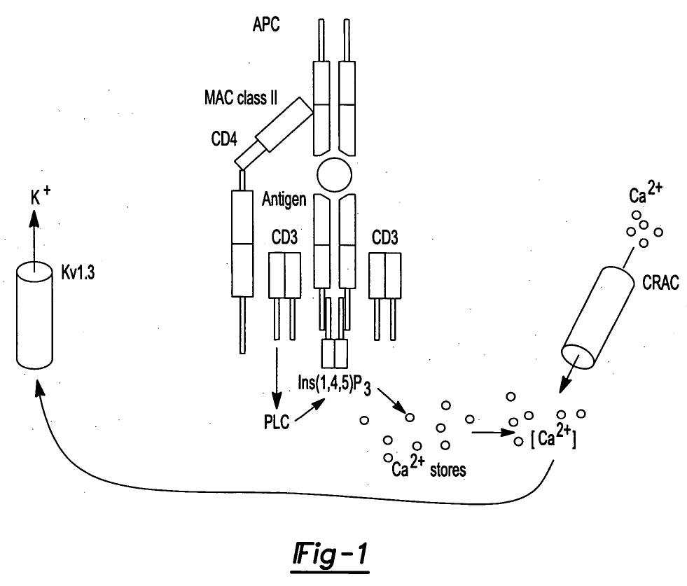

The present invention also provides for methods to monitor the progression of disease, including, but not limited to, autoimmune disease, by allowing the clinician to qualitatively and quantitatively measure the degree of autoreactivity for example, by incubating the patient's immune cells with various antigens that are indicative of disease progression. As shown in FIG. 1, when the patient's cells are responsive to discrete antigens that are known to be particularly relevant in disease expression, the patient's immune cells respond by initiating TCR/MHC receptor/antigen binding and by altering their ion channel activity. By being able to measure the Kv1.3 ion channel activity of circulating lymphocytes, the methods described herein can be used to correlate ion channel activity for example, with autoimmune diagnosis, severity and therefore, autoimmune disease.

In some embodiments methods of the present invention could be used to diagnose and detect early stages of some lymphomas. Recently, It has recently been found that integrins interact with Kv1.3 channels on lymphocyte membranes and those interactions indicate that the B1 integrin-Kv1.3 channel interaction is not static, but rather can be quite dynamic, depending upon the experimental circumstances. B1 integrin-Kv1.3 channel proximity is affected by cell adherence and the presence of K⁺ channel blockers. Thus, a direct physical interaction between B1 integrins and Kv1.3 channels may contribute to cell signaling and functions. (Artym, V.v. and Petty, H. R. (2002) J. Gen. Physiol. 120:29-38) It is believed that the interaction between voltage gated ion channels such as Kv1.3 and key metastatic proteins such as the integrin family could be exploited to develop a diagnostic assay to diagnose the presence of cancerous cells and lymphomas. Although not bound by theory, in some embodiments, the addition of lymphoma specific cell surface signaling molecules could induce activation of the B cell to proliferate and subsequently upregulate expression of the gated potassium ion channel Kv1.3. Screening for the presence of such lymphoblastic cells (uncontrolled proliferation of B cells) in the presence of proliferative signals would yield a sophisticated assay to determine the presence of lymphoblastic cells that are indicative of lymphoma. It has been recently elucidated that the changes in potassium channel expression observed in the B cell lineage parallel closely to those reported to be seen in the T-lymphocyte lineage.

The cells to be diagnosed and monitored primarily depend on what disease model is being diagnosed or monitored. In some embodiments, the disease to be diagnosed and monitored includes autoimmune diseases. As used herein the definition of autoimmune disease includes autoimmune diseases that can be characterized by cellular and humoral immune responses to epitopes on self antigens natively found in the healthy individuals. The immune system of the individual then activates an inflammatory cascade aimed at those cells and tissues presenting those specific self antigens. Without being bound to theory, it is believed that the diagnosis and monitoring of autoimmune disease according to the present invention can be carried out using hematopoietic cells (T-lymphocytes, b-lymphocytes, macrophages, monocytes, eosinophils and polymorphonuclear neutrophils. Proliferative disease can be diagnosed using hematopoietic cells (for lymphomas) biopsied cells and tissue cultured cells including for example transformed culture cells, for example Human Endothelial Kidney 293 cells (HEK293) and U937 (chronic myeloid leukemia cells) and the like.

Clinically significant autoimmune diseases include, for example, rheumatoid arthritis, multiple sclerosis, juvenile-onset diabetes, systemic lupus erythematosus, autoimmune uveoretinitis, autoimmune vasculitis, bullous pemphigus, myasthenia gravis, autoimmune thyroiditis or Hashimoto's disease, Sjogren's syndrome, granulomatous orchitis, autoimmune oophoritis, Crohn's disease, sarcoidosis, rheumatic carditis, ankylosing spondylitis, Grave's disease, and autoimmune thrombocytopenic purpura. See e.g., Paul, W. E. (1993) Fundamental Immunology, Third Edition, Raven Press, New York, Chapter 30, pp. 1033-1097; and Cohen et al. (1994) Autoimmune Disease Models, A Guidebook, Academic Press, 1994.

In some embodiments, arthritis includes several diseases with multiple etiologies are generally described as arthritides, including the following arthritic diseases Achilles tendonitis, Achondroplasia, Acromegalic, arthropathy, Adhesive capsulitis, Adult onset Still's disease, Ankylosing spondylitis, Anserine bursitis, Avascular necrosis, Behcet's syndrome, Bicipital tendonitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costosternal syndrome, CREST syndrome, Cryoglobulinemia, Degenerative joint disease, Dermatomyositis, Diabetic finger clerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arteritis, Gonococcal arthritis, Goodpasture's syndrome, Gout, Granulomatous arteritis, Hemarthrosis hemochromatosis, Henoch-Schonlein purpura, Hepatitis surface antigen disease, Hip dysplasia, Hurler syndrome, Hypermobility syndrome, Hypersensitivity asculitis, Hypertrophic osteoarthropathy, Immune complex disease, Impingement syndrome, Jaccoud's arthropathy, Juvenile ankylosing spondylitis, Juvenile dermatomyositis, Juvenile Rheumatoid arthritis, Kawasaki disease, Kienbock's disease, Legg-Calve-Perthes disease, Lesch-Nyhan syndrome, Linear scleroderma, Lipoid dermatoarthritis, Lofgren's syndrome, Lyme disease, Malignant synovioma, Marfan's syndrome, Medial plica syndrome, Metastatic carcinomatous arthritis, Mixed connective tissue disease (MCTD), Mixed cryoglobulinemia, Mucopolysaccharidosis, Multicentric reticulohistiocytosis, Multiple epiphyseal dysplasia, Mycoplasmal arthritis, Myofascial pain syndrome, Neonatal lupus, Neuropathic arthropathy, Nodular panniculitis, Ochronosis, Olecranon bursitis, Osgood-Schlatter's disease, Osteoarthritis, Osteochondromatosis, Osteogenesis, imperfecta Osteomalacia, Osteomyelitis, Osteonecrosis, Osteoporosis, Overlap syndrome, Pachydermoperiostosis, Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa Polymyalgia rheumatica, Polymyositis Popliteal cysts, Posterior tibial tendonitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid arthritis, Rheumatoid vasculitis, Rotator cuff tendonitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Subacute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective, tissue syndrome (UCTS), Urticarial vasculitis, Viral arthritis, Wegener's granulomatosis, Whipple's disease, Wilson's disease and Yersinial arthritis.

In some embodiments, the diagnosis of autoimmune disease through the use of electrophysiological analysis of ion channel activity first requires that the cells to be analyzed are first isolated from patients suffering the autoimmune disease and those from healthy controls to serve as comparisons.

In some embodiments, when the disease diagnosis is an autoimmune disease, the patient and control cell samples to be screened can include B-lymphocytes and T-lymphocytes taken from the patient or control's blood sample. The patient and control blood samples are processed identically and are measured for ion channel activity either sequentially or in parallel. The blood samples to be processed are further treated to isolate the population of cells known as peripheral blood mononuclear cells (PBMCs), which comprise lymphocytes and other white blood cells. The patient and control PMBCs are then incubated with the specific self antigen that is associated with the disease. For example in the case of rheumatoid arthritis, the PMBCs can be incubated with several known autoantigens known to be implicated in rheumatoid arthritis including, but not limited to, human collagen or structural proteins that form the components of cartilage and/or the human joint. If the patient has for example rheumatoid arthritis disease, specific autoreative effector T-cells can become activated upon presentation of the autoantigen by antigen presenting cells and illicit changes in the T-cell receptor (TCR)-mediated calcium influx, Interleukin-2 (IL-2) production and cellular proliferation. These same activated effector T-cells (CD4+ effector memory T-cells) may exhibit Kv1.3 potassium ion channel expression but at six fold higher levels than naïve, unexposed T-cells. (Liu et al., (2002) J. Exp. Med. 196:897-909).

For example, in the case of MS, myelin reactive T-cells from the blood of MS patients express highly active and increased numbers of Kv1.3 potassium channels when activated because these T-cells were subsequently identified as T (effector-memory) cells that had undergone repeated rounds of activation in vivo. In contrast, myelin reactive T-cells from healthy controls were found to possess low numbers of Kv1.3 ion channels. Hence, the identification of highly expressed Kv1.3 potassium ion channels in isolated T-cells exposed to purified myelin correlates unexpectedly well with incidence of MS. (Rus, H. et al., (2005) Proc. Natl. Acad. Sci. 102:11094-11099). The autoreactive T-cell undergoes such differentiation with concomitant upregulation of potassium voltage gated ion channels. Hence for the diagnosis and monitoring of autoimmune diseases according to the present invention, the immune cells isolated from the patient and control subjects are screened using high throughput screening assays to determine whether the isolated immune cells have upregulated the number and activity of potassium ion channels in response to challenge with autoantigens that are associated with the disease to be diagnosed or monitored.

In some embodiments, the clinician can screen the test subject and the control subject for the presence of the disorder or disease. For example, the clinician could obtain blood cells or proliferative tissue (tumor biopsy) from the subjects and controls and incubate the cells to be tested in the electrophysiological apparatus with an antigen or molecule that is known to activate the cells in question during disease. In the case of MS, patients who have the disease could be identified when their lymphocytes show an increase in ion channel activity when exposed to the specific autoantigen that is known to cause morbidity in the specific disease. In the case of MS, subjects who have T-cells that are activated in the presence of myelin basic protein would be classified as diagnosed with MS. It would generally be expected, although not bound by theory, that control subjects who do not have the disease would also not have T-cells that would respond to myelin basic protein, and thus would not have activated t-cells and not test positive for high ion-channel activity.

In the case of Systemic Lupus Erythematosus, and other arthritidae, specific autoantigens can be used in the present invention to positively diagnose the presence of those autoimmune diseases. For example, Lupus patients have a wide range of autoantibody expression, including cellular and humoral activity to the antigens La, Sm, ANCA, histone, and DNA. Although not every Lupus patient has T-cell and B-cell autoimmune responses to all of these autoantigens, the clinician can test a panel of antigen and perform the analyses in high throughput and test several hundred samples each day. Similarly, for diabetes mellitus GAD65 and insulin peptide are known autoantigens implicated in juvenile onset diabetes (type 1 diabetes).

In the case of proliferative diseases, the subject's lymphocytes are incubated with any molecule that is known to activate the proliferative cell and to cause it to become neoplastic. Activation of the cell is intimately involved with ion channel activity upregulation. In cells known to proliferate uncontrollably, it has been shown that inhibitors of ion channel activity have a dramatic ability to down regulate proliferation.

In some embodiments, the test sample can first be screened for general elevation of Kv1.3 ion channel activity. A high recording after an initial screen can form the basis for a selective screening assay for the determination of an autoimmune disorder or disease, or a non-autoimmune disorder or disease. A subject's tissue sample, for example an aliquot of blood, can be divided into two or more subsamples to be processed separately. The first subsample is processed in the initial prescreen which can include non-stimulated ion channel activity. The second subsample can then be used to specifically stimulate with a predetermined antigen or activator that is specific for a disease to be diagnosed or monitored.

In some embodiments, a control sample can include any subject not having the disease to be diagnosed or monitored.

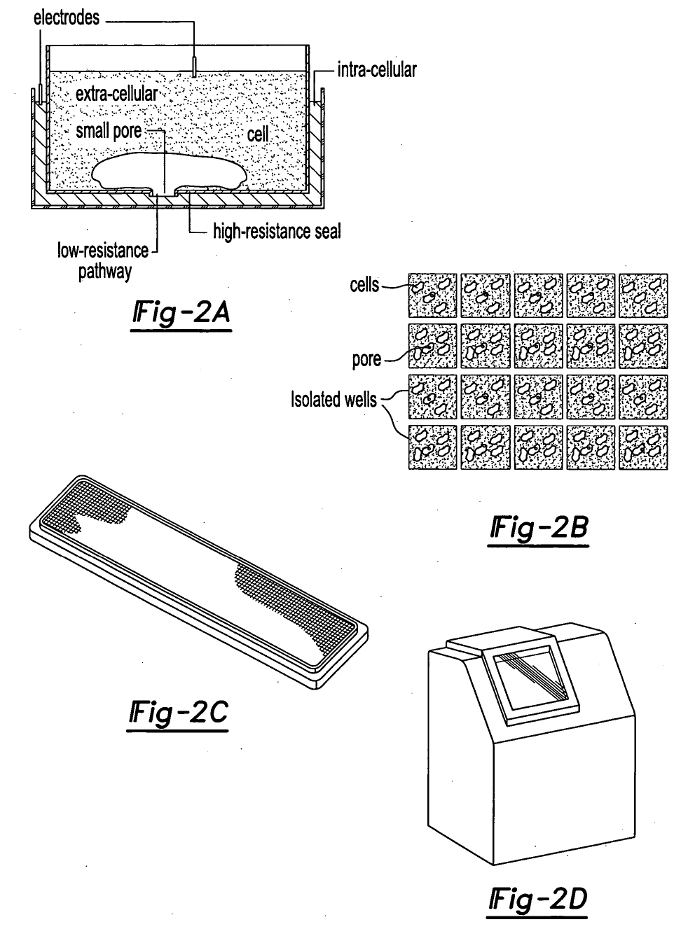

In some embodiments, the expression and activity of voltage gated potassium ion channels are measured and analyzed using high throughput electrophysiological recording equipment. It has now been unexpectedly found that arrays of cells can be screened for ion channel activity in parallel to provide a high throughput method to screen and diagnose patients with a disease known to alter the differential expression of voltage gated ion channels such as the voltage gated potassium channel Kv1.3. In some embodiments, as shown in FIG. 2, an array is provided which comprises a multiplicity of cells immobilized on a plate in contact with an electrode reading device capable of measuring ion channel activity in each cell immobilized on different extracellular potential-sensitive electrodes. In some embodiments, the array can accommodate up to 384 samples to be used for high throughput screening of ion channel activity in parallel. In some embodiments, the electrophysiological recording device is the IonWorks device manufactured by Essen Technologies, Ann Arbor Mich. This device is described in U.S. Published Patent Application No. 2003/0070923, and is incorporated by reference in its entirety.

In some embodiments, the voltage gated potassium ion channels are analyzed after incubation with a disease specific stimulus using a plate-based electrophysiology measurement platform. In some embodiments, the instrument can be an integrated platform that consists of computer-controlled fluid handling, recording electronics, and processing tools capable of voltage clamp whole-cell recordings from hundreds to thousands of individual cells. To establish a recording, the system can be a planar, multiwell substrate (including, but not limited to, a PatchPlate, including high throughput 384 well plates with at least one aperture). The system can effectively position 1 cell into a perforation separating 2 fluid compartments in each well of the substrate. Voltage control and current recordings from the cell membrane are made subsequent to gaining access to the cell interior by applying a permeabilizing agent to the intracellular side. Based on the multiwell design of the PatchPlate, voltage clamp recordings of up to 384 individual cells can be made in minutes and are comparable to measurements made using traditional electrophysiology techniques.

An integrated pipetting system can be applied for up to 2 additions of modulation agents. Typical throughput, measurement fidelity, stability, and comparative pharmacology of a voltage-dependent sodium channel for example, but not limited to (Nav1.3) and/or a voltage-gated potassium channel for example (Kv1.3) expressed in patient or control immune cells can be assayed in parallel. The high throughput device can be any device capable of biophysical and pharmacological profiling of ion channels in an environment compatible with high-capacity screening. To obtain a baseline reading of ion channel flux, an inhibitor of Kv1.3 ion channels is added to all samples. The inhibitor can be specific potassium ion channel inhibitor including Stichodactyla helianthus peptide (ShK). The readings are taken before and after the addition of the inhibitor specific to Kv1.3 to obtain a net ion channel current that can be compared to other samples for determination of diagnosis or for monitoring disease.

In some embodiments, the high-throughput electrophysiological apparatus and fluorescence based assays can measure ligand gated channels. In some embodiments, ion channels are gated by extracellular ligands. However, in some embodiments, ion channels are gated by intracellular ligands. In some embodiments examples of ligand channels could include Acetylcholine (ACh) and Gamma amino butyric acid (GABA) ligands to open Cl− channels. The binding of the neurotransmitter acetylcholine at certain synapses opens channels that admit Na+ and initiate a nerve impulse or muscle contraction.

In some embodiments, ion channel activity data can also be measured and utilized when information concerning the progression of the disease in a patient having the disease is desirable. In such instances, the patient can be monitored with respect to disease severity or degree of remission or recuperation by measuring the degree of ion channel activity in response to challenge with for example, a disease associated autoantigen.

In some embodiments, a patient suffering for example, from an autoimmune disease can be monitored by assaying their autoimmune ion channel activity in the presence of autoantigen associated with the disease and correlating such ion channel activity with disease progression. In practice, the subject would have periodic withdrawals of sample tissue. In some embodiments, blood samples could be drawn on a patient having the autoimmune disease every month, and assayed for ion channel activity. Since the use of channel inhibitors have been shown to reduce proliferative disease it is believed that channel activity correlates closely with active disease. Ion channel activity could be employed to determine whether a specific patient's treatment protocol is effective. For example, patients suffering from an autoimmune disease can be diagnosed as having the autoimmune disease after measuring the patient's ion channel activity in response to challenge with an autoantigen associated with such an autoimmune disease. After commencing a treatment regimen, the patient can at some predetermined interval after commencement of treatment have their immune cells purified and tested again by incubating their immune cells in the presence of the disease associated autoantigen. If the treatment is successful in ameliorating the disease, the ion channel activity and number of ion channels produced by the patient's immune cells in response to the autoantigen will have abated or diminished. Optionally, the progression of disease in the patient and the efficacy of treatments to overcome the disease can be evaluated by measuring the ion channel activity of the patient's immune cells in a high throughput electrophysiological assay when referenced to normal healthy controls.

In some embodiments the disease to be diagnosed or monitored can be an autoimmune disease. In some embodiments the disease can relate to proliferative disorders where the cell has forgone normal cell programming but has embarked on abnormal cell growth.

The invention will now be described with reference to a non-limiting example.

### EXAMPLE 1

Preparation of T-Cells for Use in Diagnostic and Monitoring Assays

Whole blood samples can be collected according to the standard phlebotomy procedures used by the clinician. In some embodiments, samples to be assayed can include healthy matched controls and samples provided by patients suspected of presenting MS or patients with established symptoms of MS. Peripheral Blood Mononuclear Cells (PBMCs) can be isolated using established centrifugation techniques. In some embodiments, PBMCs can be isolated from other components using density gradient centrifugation. In some embodiments, whole blood can be diluted 1:2 in physiologically acceptable buffer, for example, Dulbecco's Phosphate Buffered Saline (DPBS) and layered over an appropriate density gradient solution, such as Histopaque-1119 (Sigma Aldrich, St Louis, Mo.) as per manufacturers instructions. The mixture can then be centrifuged to separate blood into its constituent bands. In some embodiments, the bands containing plasma and PMBCs are collected in separate tubes. The platelets in the plasma band can further be removed by centrifugation. The plasma can then be added to the sample containing the PBMCs and the mixture can then be transferred into a sterile culture flask.

The specific antigen that will elicit the activation of autoreative T-cells to the host's myelin protein thus allowing for a diagnosis of MS is introduced to the control and test samples. In the case of the present example, human myelin basic protein (MBP) can be added (hMBP; 10 μg/mL, Sigma Aldrich, St. Louis, Mo.) to the flasks containing plasma and PBMCs. The PBMCs also contain antigen-presenting cells (APCs), including monocytes, macrophages and B-cells. Upon exposure to MBP, APC's specific for MBP present MBP antigen along with co-stimulatory signals to activate MBP-specific T-cells (as in the case with MS patients, but not normal healthy controls). The flasks can be incubated for 6 to 12 hours at 37° C. to allow for stimulation of MBP-specific T-cells. After the incubation negative selection can be applied to isolate and purify specific T-cell subsets that will be tested for potassium ion channel activity. In the present example, RosetteSep (StemCell Technologies Inc., Vancouver, BC Canada) enrichment cocktail can be added to isolate either CD4+ or CD8+ T-cells from all other non-CD8+ or nonCD4+ T-cells. The CD4+ T-cells or CD8+ T-cells are concentrated by centrifugation and washed twice before analysis of potassium ion channel activity with the high throughput electrophysiology system.

Diagnosis and Monitoring Multiple Sclerosis

Certain T-lymphocytes (i.e. activated effector memory T-cells) in the blood of patients with MS show an elevated activity of the Kv1.3 ion channel. As previously shown, T-cell activation leads to an elevated ion channel activity in for example, in MS patients as a consequence of repeated in vivo stimulation of pathogenic, myelin-reactive T-cells by the antigens that elicit the autoimmune reaction, e.g. myelin basic protein (MBP) in the case of MS. During this autoimmune-induced stimulation, the expression levels of Kv1.3 ion channels can increase dramatically from approximately 250 to 1,500 channels per cell.

In vitro experiments have showed that only a specific subset of T-cells, the myelin-reactive effector memory T-cells, react in such a strong way to stimulation with myelin antigens (e.g. hMBP), other myelin-reactive subsets of T cells, which are present in healthy individuals (e.g. naive and central memory cells), in contrast, require 7-10 stimulation cycles with MBP before acquiring the effector memory phenotype and before up-regulating their Kv1.3 activity.

As discussed above, the difference in response to MS-specific in vitro stimulation by MBP suggests that it can be possible to perform a diagnostic assay specific for MS based on Kv1.3 by proceeding in two steps. In the first step a preparation of T cells from patients with questionable MS diagnosis could be screened for increased Kv1.3 activity. If this test would turn out positive then a second round of specific in vitro stimulation of the T-cells from this patient with MBP could establish whether the increased Kv1.3 activity is in fact caused by MS or may be caused by other infectious or other autoimmune diseases. A recent article showed that only the T-cells from MS patients increased their Kv1.3 activity upon exposure to MBP, T cells from control patients did not.

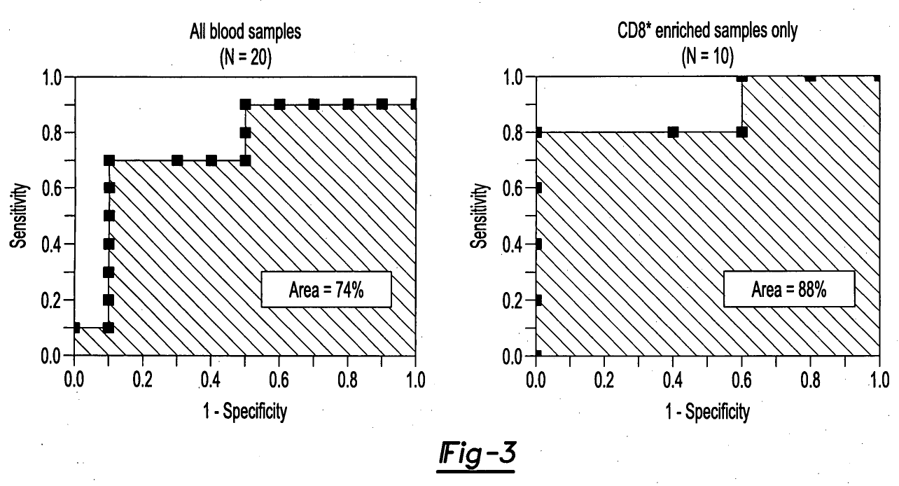

T-cells from control patients with other autoimmune diseases than MS did not respond to stimulation with MBP as shown in FIG. 3. Conversely, activating T cells from MS patients with known antigens of other autoimmune diseases than MS did not increase the Kv1.3 activity in these cells. In the context of a diagnostic assay for MS, these findings can be applied to a diagnostic assay for the diagnosis of MS because the T-cells of MS patients are repeatedly stimulated with myelin antigens in vivo; these patients have circulating myelin-reactive T-cells of the effector memory type. If activated (either by the autoimmune pathogenesis of MS in vivo or by adding MBP and antigen presenting cells (in vitro), these T-cells from MS patients display a strongly increased Kv1.3 ion channel activity, whereas healthy controls do not develop the myelin-reactive effector memory T cell phenotype; the it myelin-reactive T cells remain in the naive or central memory state upon in vitro stimulation and consequently express approximately six-fold lower Kv1.3 activity compared to the T-cells of MS patients.

As practiced in this example, it is believed that the difference in ion channel activity found between healthy and MS patients can offer a strategy for a functional and specific diagnostic assay for many autoimmune diseases including MS. Kv1.3 channels cluster at the site of interaction between cytotoxic T-cells and their target cells. Activated autoreactive effector memory T-cells contribute to MS by migrating to inflamed tissues where they secrete interferon-γ (IFG) and tumor necrosis factor-α (TNFα). Adoptive transfer of Kv1.3 high rat memory T-cells into native recipients has been shown to cause severe experimental autoimmune encephalomyelitis (EAE), a model for MS. The pathologically elevated Kv1.3 ion channel activity in effector memory T-cells therefore plays a role in autoimmune attack on the myelin sheets of nerve cells of MS patients.

The present invention requires the use of a high throughput instrument for electrophysiological measurements that makes it possible to record from 384 cells in parallel (FIG. 2). This high-throughput screening instrument, called IonWorks HT, was developed at a Biotech company, Essen Instruments Inc., Ann Arbor, Mich., USA.

The IonWorks instrument allows, the operator, to perform functional tests on ion channels (i.e. measuring the physiological function of ion channel proteins, namely the regulation of the ion flux across cell membranes in high throughput. In the clinical and research setting, electrophysiological recordings are usually performed by an experienced scientist on one individual biological cell at a time. Screening for specific ion channel activity in blood cells for example was therefore not even imaginable. The IonWorks HT electrophysiological measurement apparatus can be operated according to manufacturer's instructions.

Modern high throughput electrophysiology technology is capable of detecting ion channel currents that are characteristic for patients with MS; the same blood cells from control subjects showed significantly lower ion channel activity part of both Lower trace after blocking the ion channel.

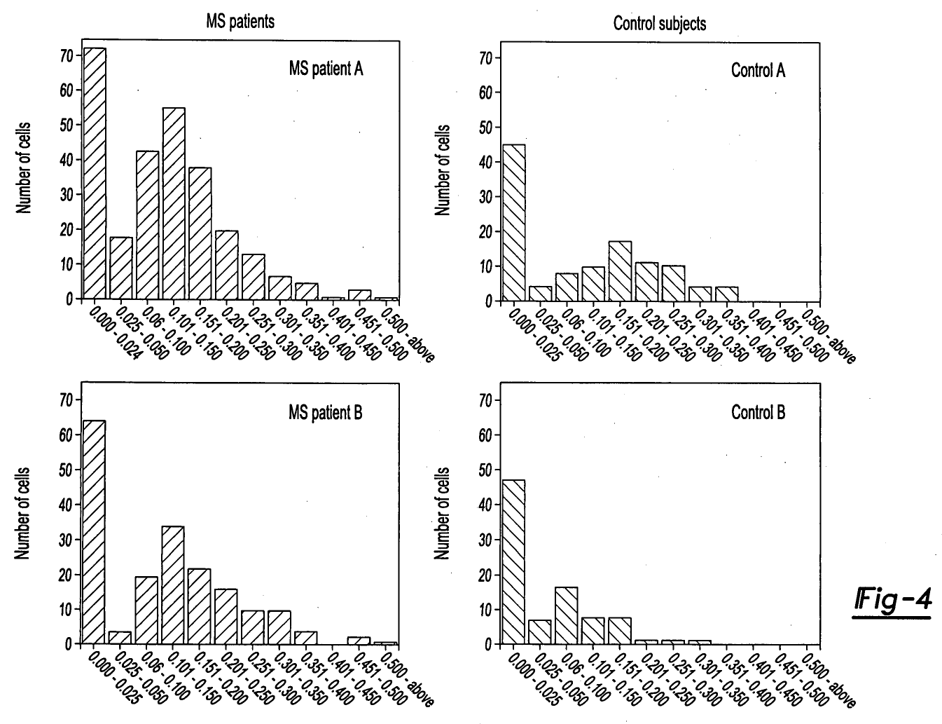

The diagnostic value of automated ion channel activity analysis technology lies in the possibility to analyze up to 384 blood samples in parallel. This capability makes it possible to identify subpopulations of blood cells with reliable statistics. For instance, the T-lymphocytes (CD4+ and CD8+) that express Kv1.3 ion channels in MS patients constitute only a fraction of all white blood cells. (Wulff, H. et al., (2003) Curr. Opin. Drug Discov. Devel. 6:640-647). FIG. 4 illustrates the statistic of ion channel activity from preparations of white blood cells from MS patients compared to control healthy subjects. As shown in FIG. 4, it was unexpectedly found that in MS patients typically 3-16% of blood cells showed Kv1.3 ion channel activity above 150 nA·msec, whereas the percentage of blood cells above this threshold was 0% for control subjects.

In some embodiments, larger volumes of blood samples (20 mL instead of 2 mL) from MS patients and healthy controls can be utilized to yield a larger sample of autoreative T-cells. In some embodiments, the larger patient and control blood samples enable specific selection of T-cells that express Kv1.3 channels in large numbers upon stimulation, these subsets can include, but are not limited to CD 4+ and CD8+ T-cells.

In some embodiments, purified subsets of either CD4+ or CD8+ cells can be used in the methods described herein to identify the most suitable subset of T-cells for diagnosis of MS. Table 1. Shows a comparison of the total white blood cells with Kv1.3 activity obtained from different populations. CD8+ cell enrichment from a larger blood volume increased the number of cells with Kv1.3 activity in patients with MS from an average of 9 cells (2 mL of blood) to an average of 142 cells (20 mL of blood). Selecting for CD8+ cells also increased the number of cells with Kv1.3 activity in healthy controls (from 7 to 70 cells), which is no surprise considering that Kv1.3 channels are part of the physiologic immune response of the body. The significant result, however, is the difference in the number of activated T-cells from MS patients compared to healthy controls. On average the number of activated T-cells in MS patients as compared to healthy controls can be double (142 versus 70).

Data Analysis

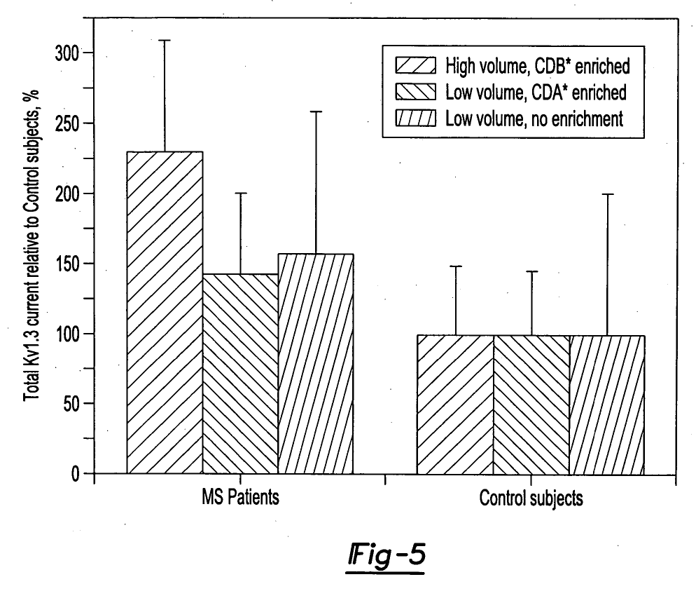

In order to analyze the data from the IonWorks reader in an automated procedure, an algorithm is developed. For each analyzed blood sample, the algorithm first identifies the recordings with seal resistances of at least 75 MΩ. This condition removed unreliable data from cells that might not have sealed sufficiently to the micropore to guarantee stable recording conditions. The algorithm then determines the maximum current amplitude of the specific Kv1.3 current (as defined by the maximum difference between the current before and after blockage of all Kv1.3 channels with the ShK toxin). These current amplitudes are then plotted in current histograms such as the ones shown in FIG. 5. These histograms yield the number of cells with Kv1.3 activity for MS patients and controls. These data are then compiled through the algorithm and results are used to create the total numbers of cells with Kv1.3 activity as shown in Table 1.

FIG. 5, refers to electrophysiological analysis of T-cells from MS patients and Control patients. MS patient not only had a greater number of T-cells with elevated Kv1.3 activity, these T-cells also had on average a higher magnitude of Kv1.3 current. This observation suggested using the sum of all Kv1.3 currents per blood sample as the metric for distinguishing between MS patients and Controls. Table 2 summarizes the results of the total Kv1.3 current analysis for different preparations of T-cells. Using CD8+ T-cells enriched from 20 mL of whole blood, the method described herein found a greater than two-fold difference between the average total Kv1.3 current in blood samples from MS patients compared to samples from control subjects.

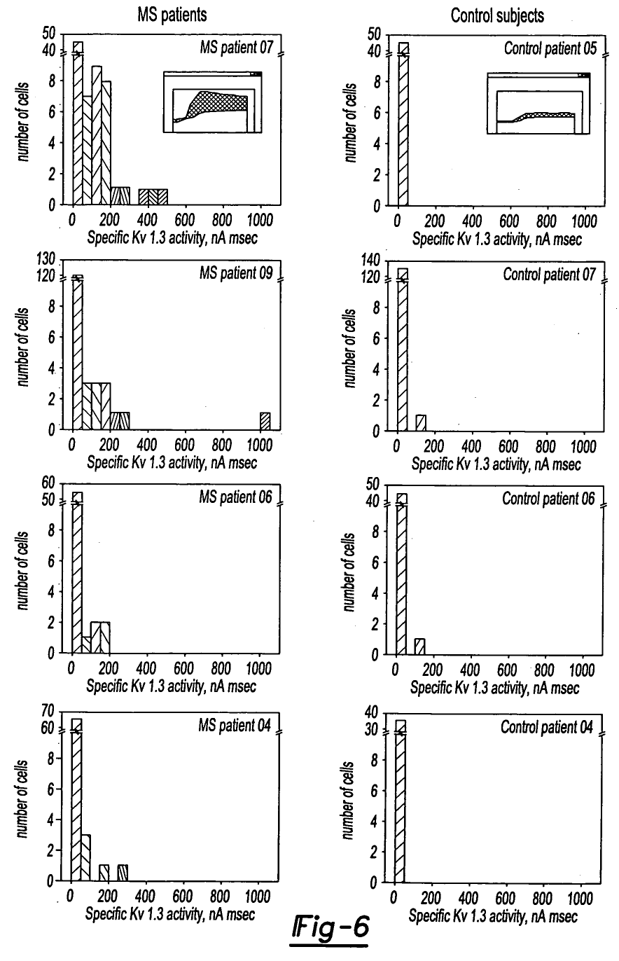

FIG. 6, refers to comparisons between normalized total currents from different preparations. As described herein, the method can be developed using T-cells enriched from a large sample of blood, which can be safely obtained as for example during a routing blood test. In addition to CD8+ T-cells being useful for the methods described herein, CD4+ T-cells can also be employed in the methods of the present invention. CD4+ T-cells can be used preferentially when specific disease states involve enhanced activity of ion channels expressed on CD4+ T-cells rather than the CD8+ T-cell types. In some embodiments, the methods described above can be utilized with test and control blood samples requiring the further step of purifying CD4+ T-cells in addition to CD8+ T-cells using methods commonly used in the art (for example, magnetic bead separation using CD4 specific antibodies, or cell sorting using flow cytometry gated for CD4+ T-cells excluding other lymphocytes).

Specificity and Sensitivity of the Assays

The assays and methods of the present invention were evaluated to determine the level of sensitivity and specificity of ion channel measurements between normal and diseased T-cells. In some embodiments, tests can be performed to ensure that the specificity and sensitivity of the screening assays allows the practicing clinician to differentiate between normal and diseased states. One of the questions to be resolved is whether the screening assays and methods described herein produce unacceptably high numbers of false positives or false negatives.

In some embodiments, the analysis of whether the screening assays are sufficiently specific and sensitive requires the use of Receiver Operating Characteristics (ROC) curves commonly used in evaluating the quality of diagnostic assays. ROC curves provide information as to the number of true positives (TP) and true negatives (TN), as well as the number of false positives (FP) and false negatives (FN) for a given diagnostic assay when varying thresholds are applied. Based on the ROC curves, the sensitivity of a diagnostic assay can be calculated as the probability that the test correctly classifies a positive result, wherein:
Sensitivity=TP/(TP+FN) Eq. 1
Similarly, the ROC curve also identifies the specificity of a diagnostic test, defined as the probability that the test correctly classifies a negative result, wherein:
Specificity=TN/(TN+FP) Eq. 2
In order to determine the quality of a diagnostic assay, an ROC curve can be generated by plotting the sensitivity (Eq. 1) over (1—specificity (Eq. 2)) for varying thresholds. (Nielsen, N. E. et al., (2000) J. Intern. Med. 247:43-52). The area under the ROC curve defines the statistical quality of the test. An area of 1 (100%) represents an ideal test (meaning that there is a value for the threshold where both the sensitivity and specificity would be 100%). A diagnostic assay that results in an area under the ROC curve of 0.9 (90%) is considered outstanding (for comparison, the area under the ROC curve for digital mammography is about 0.85 for women over the age of 50).

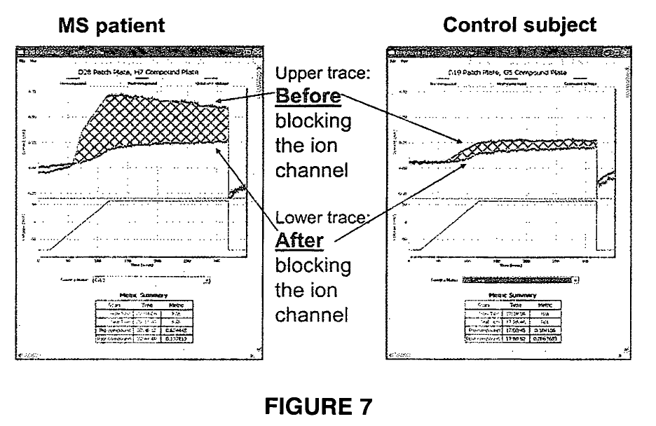

The ROC curve for the diagnostic assays described in the present invention was constructed from all of the data gathered from the Kv1.3 screening experiments using blood samples from MS patients and healthy controls. FIG. 7. Represents the ROC curve constructed using the data gathered using Kv1.3 as the statistical data points. An area of 0.74 was obtained when all experiments were included (blood samples without enrichment for CD8+ T-cells) in the construction of the ROC curve (total of 10 MS patients and 10 normal controls). The quality of the data improved significantly, as expected, when the samples of the MS patients (5) and normal controls (5) were enriched for CD8+ T-cells. The resulting ROC curve generated an area of 0.88, better than the quality of digital mammography used presently.

The ROC curve analysis for enriched CD8+ T-cells as shown in FIG. 7, yielded an optimum value for the total Kv1.3 current of 13.5-16.3 nA. In some embodiments the inventors were surprised to find that the results of the diagnostic assays described herein showed that that the assay is capable of detecting at least four true positives, one false positive, five true negatives and zero false negatives out of ten patient samples tested.

In conclusion, the average results obtained from CD8+ enriched T-cell fractions from MS patients showed 230% of the total Kv1.3 current activity of the healthy control subjects tested in parallel. FIG. 7 shows that the diagnostic quality of an MS test based on Kv1.3 gated ion channels using enriched CD8+ T-cells can be very high. In non-limiting embodiments, similarly high diagnostic qualities are expected when diagnosing and/or monitoring other diseases with other isolated cells known to be implicated and/or affected in the disease. The methods used to assay Kv1.3 ion channel activity described herein are automated and cell preparation (either biopsy, primary culture or blood) may involve established cell separation techniques that can be performed routinely by the clinician with minimal preparation, set up time and expense.

What is claimed is:

1. A process for diagnosing a disorder or disease comprising:
A) providing (1) at least one sample of tissue from a subject and (2) an electrophysiological measurement apparatus that is capable of high throughput ion flux measurement;
B) obtaining at least one voltage-gated ion channel-containing (ICC) structure from the at least one sample of tissue or from a subsample thereof;
C) making an electrophysiological measurement of the ICC structure, using the electrophysiological measurement apparatus, operated in a high-throughput mode, by (1) applying a voltage across the ICC structure to obtain a potentiated ICC structure, and (2) measuring ion flux across voltage-gated ion channels of the potentiated ICC structure to obtain a test result;
D) comparing the test result to a standard or control result obtained, under identical conditions, for ICC structure of a nonpathological version of the tissue, to obtain a difference; and
E) using said difference to make a diagnosis of a disorder or disease, the disorder or disease being an autoimmune disorder or disease or a non-autoimmune, proliferative disorder or disease.

2. The process according to claim 1, wherein said ICC structure is a lymphocyte ICC structure and said step (B) of obtaining the structure involves contacting the lymphocytes with at least one antigen or epitope.

3. The process according to claim 1, wherein said electrophysiological measurement apparatus comprises a high throughput patch plate electrophysiological measurement apparatus.

4. The process according to claim 1, wherein the electrophysiological measurement apparatus is capable of analyzing about 200 or more samples concurrently.

5. The process according to claim 1 wherein the voltage-gated ion channels comprise voltage-gated potassium ion channels.

6. The process according to claim 5, wherein the voltage-gated potassium ion channels comprise Kv1 type voltage-gated potassium ion channels.

7. The process according to claim 5, wherein the Kv1 type voltage gated potassium ion channel is Kv1.3 voltage-gated potassium ion channels.

8. The process according to claim 1, wherein the tissue sample (A)(1) comprises lymphocytes and the ICC structure (B) being a lymphocyte ICC structure, wherein said process further comprises:
splitting said tissue sample (A)(1) into at least two subsamples,
F) contacting at least one of the subsamples with at least one antigen or epitope to obtain a challenged subsample, and
G) performing steps (B) through (E) using the challenged subsample(s).

9. The process according to claim 1, wherein said disorder or disease comprises any of the autoimmune diseases.

10. The process according to claim 9, wherein said autoimmune disease comprises any of arthritis, systemic lupus erythematosus, Sjogren's syndrome, type 1 diabetes mellitus, graves disease, celiac disease, multiple sclerosis, Guillain-Barre, Hashimoto's thyroiditis, chronic graft versus host disease, and Crohn's disease.

11. The process according to claim 10, wherein said arthritis disease comprises any of Achilles tendonitis, Achondroplasia, Acromegalic, arthropathy, Adhesive capsulitis, Adult onset Still's disease, Ankylosing spondylitis, Anserine bursitis, Avascular necrosis, Behcet's syndrome, Bicipital tendonitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costosternal syndrome, CREST syndrome, Cryoglobulinemia, Degenerative joint disease, Dermatomyositis, Diabetic finger clerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arteritis, Gonococcal arthritis, Goodpasture's syndrome, Gout, Granulomatous arteritis, Hemarthrosis hemochromatosis, Henoch-Schonlein purpura, Hepatitis surface antigen disease, Hip dysplasia, Hurler syndrome, Hypermobility syndrome, Hypersensitivity asculitis, Hypertrophic osteoarthropathy, Immune complex disease, Impingement syndrome, Jaccoud's arthropathy, Juvenile ankylosing spondylitis, Juvenile dermatomyositis, Juvenile Rheumatoid arthritis, Kawasaki disease, Kienbock's disease, Legg-Calve-Perthes disease, Lesch-Nyhan syndrome, Linear scleroderma, Lipoid dermatoarthritis, Lofgren's syndrome, Lyme disease, Malignant synovioma, Marfan's syndrome, Medial plica syndrome, Metastatic carcinomatous arthritis, Mixed connective tissue disease (MCTD), Mixed cryoglobulinemia, Mucopolysaccharidosis, Multicentric reticulohistiocytosis, Multiple epiphyseal dysplasia, Mycoplasmal arthritis, Myofascial pain syndrome, Neonatal lupus, Neuropathic arthropathy, Nodular panniculitis, Ochronosis, Olecranon bursitis, Osgood-Schlatter's disease, Osteoarthritis, Osteochondromatosis, Osteogenesis, imperfecta Osteomalacia, Osteomyelitis, Osteonecrosis, Osteoporosis, Overlap syndrome, Pachydermoperiostosis, Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa Polymyalgia rheumatica, Polymyositis Popliteal cysts, Posterior tibial tendonitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid arthritis, Rheumatoid vasculitis, Rotator cuff tendonitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Subacute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective, tissue syndrome (UCTS), Urticarial vasculitis, Viral arthritis, Wegener's granulomatosis, Whipple's disease, Wilson's disease and Yersinial arthritis.

12. The process according to claim 1, wherein said non-autoimmune proliferative disorder or diseases comprises any of the melanomas, lymphomas, neoplasms, and the like.

13. The process according to claim 1 wherein the sample of tissue comprises any one of: whole blood; white blood cells; lymphocytes, cell fragments, and plasma membranes thereof.

14. The process according to claim 13, wherein white blood cells comprise T-lymphocytes and B-lymphocytes.

15. The process according to claim 14, wherein said T-lymphocytes further comprise CD4+ and CD8+ T-cells.

16. The process according to claim 1, wherein said electrophysiological measurement comprises high throughput electrophysiological measurements taken before and after adding an ion channel blocker.

17. A process according to claim 1, wherein said voltage-gated ion channel-containing structure comprises the potassium voltage gated ion channel Kv1.3.

18. A process for monitoring a disease comprising:
(A) providing (1) at least one sample of tissue from a subject at time X, (2) at least one sample of tissue from the same subject at time Y subsequent to time X, and (3) an electrophysiological measurement apparatus that is capable of high throughput ion flux measurement;
(B) obtaining at least one voltage-gated ion channel-containing (ICC) structure from each of the time X and time Y tissue samples or from subsamples thereof;
(C) making an electrophysiological measurement of the obtained time X and time Y ICC structures, under substantially identical conditions, using the electrophysiological measurement apparatus, operated in high-throughput mode, by applying a voltage across each ICC structure to obtain a potentiated ICC structure, and (2) measuring ion flux across voltage-gated ion channels of the potentiated ICC structure to obtain a test result;
(D) comparing the electrophysiological measurement from the time X sample(s) with that of the time Y sample(s), or both that and comparing these with a standard or control result derived under substantially identical conditions, for ICC structure of a nonpathological version of the tissue to obtain a difference; and
(E) using said difference to determine the stage of development of, the stage of progression of, or the status of a disease or disorder in the subject the disorder or disease being an autoimmune disorder or disease or a non-autoimmune, proliferative disorder or disease.

19. The process according to claim 18, wherein said ICC structure is a lymphocyte ICC structure and said step (B) of obtaining the structure involves contacting the lymphocytes with at least one antigen or epitope.

20. A process according to claim 18, wherein the time between time X and time Y is about one week to about one year.

21. The process according to claim 18, wherein said electrophysiological measurement apparatus comprises a high throughput patch plate electrophysiological measurement apparatus.

22. The process according to claim 18, wherein the electrophysiological measurement apparatus is capable of analyzing about 200 or more samples concurrently.

23. The process according to claim 18 wherein the voltage-gated ion channels comprise voltage-gated potassium ion channels.

24. The process according to claim 23, wherein the voltage-gated potassium ion channels comprise Kv1 type voltage-gated potassium ion channels.

25. The process according to claim 23, wherein the Kv1 type voltage gated potassium ion channel is Kv1.3 voltage-gated potassium ion channels.

26. A process for evaluation of a response to a therapeutic or diagnostic treatment of a disorder or disease, comprising:
(A) providing (1) (a) at least one first sample of tissue from a subject diagnosed with an autoimmune disorder or disease or non-autoimmune proliferative disorder or disease, taken prior to treatment, and (b) at least one second sample of tissue taken from the same subject after administration of a therapeutic or diagnostic agent thereto, (2) an electrophysiological measurement apparatus that is capable of high throughput ion flux measurement;
B) obtaining at least one voltage-gated ion channel-containing (ICC) structure from each of the tissue samples or from a subsample thereof;
C) making an electrophysiological measurement of each of the obtained ICC structures, using the electrophysiological measurement apparatus, operated in a high-throughput mode, by (1) applying a voltage across the ICC structure to obtain a potentiated ICC structure, and (2) measuring ion flux across voltage-gated ion channels of the potentiated ICC structure to obtain a first test result for the first sample and a second test result for the second sample;
D) comparing the first and second test results to each other, or both to each other and further to a standard or control result obtained, under substantially identical conditions, for ICC structure of a nonpathological version of the tissue, to obtain a difference; and
E) using said difference to evaluate a response to the therapeutic or diagnostic treatment, the disorder or disease being an autoimmune disorder or disease or a non-autoimmune, proliferative disorder or disease.

27. The process according to claim 26, wherein said electrophysiological measurement apparatus comprises a high throughput patch plate electrophysiological measurement apparatus.

28. The process according to claim 26, wherein the electrophysiological measurement apparatus is capable of analyzing about 200 or more samples concurrently.

29. The process according to claim 26 wherein the voltage-gated ion channels comprise voltage-gated potassium ion channels.

30. The process according to claim 29, wherein the voltage-gated potassium ion channels comprise Kv1 type voltage-gated potassium ion channels.

31. The process according to claim 29, wherein the Kv1 type voltage gated potassium ion channel is Kv1.3 voltage-gated potassium ion channels.

32. The process according to claim 26, wherein said second sample is taken during a course of said treatment.
